(19) United States
(12) Patent Application Publication
(10) Pub. No.: US 2012/0277763 A1
(43) Pub. Date: Nov. 1, 2012

(54) DYNAMIC ABLATION DEVICE

(75) Inventors: Elliott E. Greenblatt, Cambridge, MA (US); Karen I. Trovato, Putnam Valley, NY (US); Thomas J. Naypauer, JR., Cleveland Heights, OH (US); Sunny Virmani, Twinsburg, OH (US)

(73) Assignee: KONINKLIJKE PHILIPS ELECTRONICS N.V., EINDHOVEN (NL)

(21) Appl. No.: 13/516,757

(22) PCT Filed: Dec. 21, 2010
(86) PCT No.: PCT/IB2010/055995

§ 371 Date: Jun. 18, 2012

Related U.S. Application Data
(60) Provisional application No. 61/290,973, filed on Dec. 30, 2009.

(51) Int. Cl. A61N 5/01; A61B 19/00

(52) U.S. Cl. 606/130; 607/101

(57) ABSTRACT

In an interventional ablation therapy planning system (10), an imaging system (30) generates an image representation of a target volume located in a patient. A segmentation unit (36) segments a planned target volume (42) of the target volume which is to receive the ablation therapy. A planning processor (40) which generates an ablation plan with one or more ablation zones (44, 48, 50, 52) which cover the entire planned target volume (42) with ablation therapy, each ablation zone has a predetermined ablation volume, the predetermined ablation zone being defined by moving an ablation probe (12) during ablation. A robotic assembly guides or controls the ablation probe (12) along a non-stationary motion path which is defined by a trajectory, velocity and/or acceleration, and a rotation to apply ablation therapy to the target volume according to the predetermined ablation zone(s).

The present application relates to ablation therapy planning. It finds particular application in image based planning and guidance of interventional radio frequency ablation.

In radio frequency ablation (RFA) techniques, a radio frequency probe comprised of an insulated lead and an exposed electrode is used to heat surrounding tissue above 50 degrees Centigrade. At this temperature, proteins are permanently denatured, cell functions are destroyed, and histological damage can be seen. RFA has produced promising results in the treatment and management of unrespectable tumors. Typically, the probe is connected to a radio frequency generator and receives approximately 460-500 kHz AC power for a predetermined time, e.g. approximately 15 minutes or other suitable time period which generates an ablation zone that generally resembles a sphere or ellipsoid. A planned target volume (PTV) includes the tumor plus a margin, generally around 1 cm. If the PTV is larger than the ablation zone, then multiple ablations can be used to cover the PTV. Under current practice, a surgeon makes a mental note with the location of the lesion and inserts the probe under guidance from image-based or other tracking methods. Oftentimes, the probe can be visualized easily but the target volume may not always be discernable. Moreover, because each probe is very expensive, a surgeon is often deterred from employing multiple probes with various sized ablation zones, in favor of attempting to ablate the target volume using a minimum number of probes, typically only one.

When a PTV can be covered by a single ablation zone, the tumor recurrence rate following RF ablation is comparable to that of tumors treated surgically. However, for larger PTV's, which exceed a size that cannot be successfully covered by a single ablation, the recurrence rate following RF ablation increases. This is believed to be due to incomplete ablation of the PTV, since leaving any untreated portion oftentimes causes an aggressive recurrence.

The mental exercise for covering a PTV with multiple ablations is complex. For example, a spherical PTV that is 1.7 times larger than the size of a unit ablation zone requires over 14 ablations. Each ablation, typically takes about 15 minutes, and not only adds to the surgical and anesthesia time and cost, but also poses a greater risk to the patient. Ablating near critical structures poses an even greater risk because inadvertent damage from operator error, organ motion, improper planning, or the like can cause serious injury to the patient.

Success of RF ablation procedures relies on accurate deposition of the thermal dose into the cancerous lesion, while also sparing healthy tissue in order to minimize side effects. Difficulties and potential errors arise when surgeons attempt to mentally visualize the planned target volume in a three-dimensional space while controlling the probe such that it accurately reaches the intended location(s). Tumor shapes and sizes are often irregular and do not match the spherical or ellipsoidal ablation zone of the probe thus complex three-dimensional calculation and visualizations are employed to determine a coverage plan. Perfect coverage of the PTV is unlikely because of geometric complexity of the PTV, difficulties in maneuvering the probe to precise locations, and relatively long ablation times. Current treatment methods rely heavily on approximation, which leaves open the possibility of under-treatment or over-treatment. Under-treatment can result in aggressive recurrence of the tumor which can ultimately lead to death. Over-treatment causes two problems: collateral damage and long procedural time. Collateral damage occurs when the size of the ablation zone causes excessive ablation of healthy tissue. Long procedural times result when the estimated number of ablations is large, making the procedure intolerably long for the patient, typically due to anesthesia risk.

The present application provides a new and improved dynamic ablation system and method, which overcomes the above-referenced problems and others.

In accordance with one aspect, a method for interventional ablation therapy planning is presented. An image representation of a target volume in a subject is generated from which a planned target volume to receive ablation therapy from an ablation probe is determined. The planned target volume defines a region, which includes the target volume of the subject. An ablation plan is generated to cover the planned target volume. The ablation plan includes one or more ablation zones, which cover the entire planned target volume with ablation therapy. Each ablation zone has a predefined ablation volume, which is defined by moving an ablation probe during therapy.

In accordance with another aspect, an interventional ablation therapy planning system is presented. An imaging system generates an image representation of a target volume in a subject. A segmentation unit determines a planned target volume from the image representation, which is to receive ablation therapy. The planned target volume defines a region, which includes the target volume. A planning processor generates an ablation plan. The ablation plan includes one or more ablation zones that cover the entire planned target volume with ablation therapy. Each ablation zone has a predetermined ablation volume, which is defined by moving an ablation probe during ablation.

According to another aspect, a method for generating an ablation zone using an ablation probe is presented. The method includes determining a trajectory of the ablation probe, determining acceleration non-constant velocity profile of the ablation probe along the determined trajectory, and applying ablation therapy while the ablation probe travels along the determined trajectory at the determined non-constant velocity profile.

One advantage resides in minimizing therapy duration.

Another advantage is that the number of ablations to ablate a planned target volume is reduced.

Another advantage is that critical regions are identified and avoided during therapy.

Another advantage resides in increasing the accuracy of covering the planned target volume with ablative therapy.

Another advantage resides in minimizing overlap of ablation zones.

Still further advantages of the present invention will be appreciated to those of ordinary skill in the art upon reading and understand the following detailed description.

The invention may take form in various components and arrangements of components, and in various steps and arrangements of steps. The drawings are only for purposes of illustrating the preferred embodiments and are not to be construed as limiting the invention.

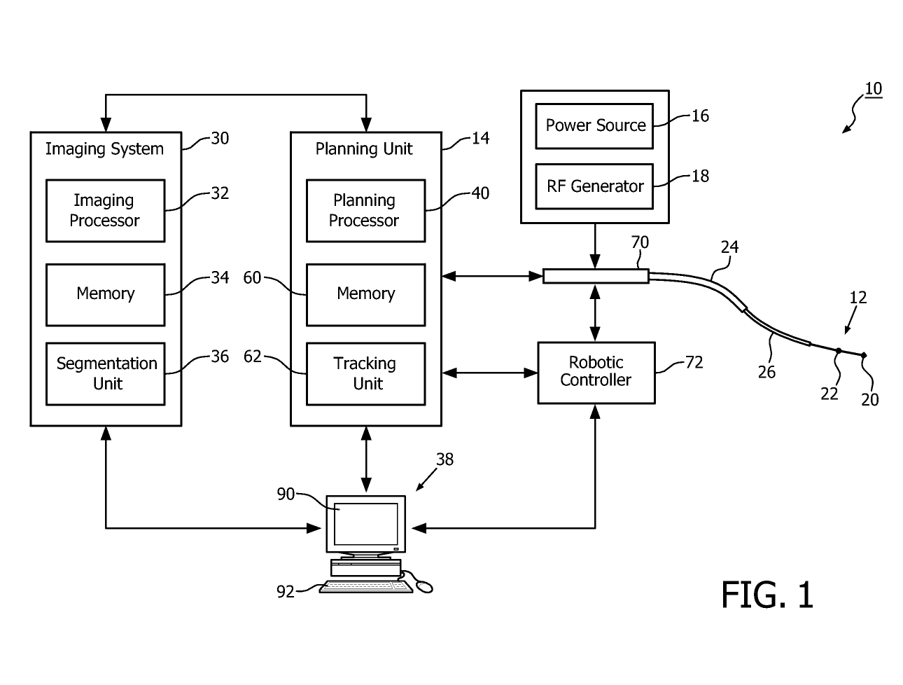

FIG. 1 is a diagrammatic illustration of an interventional radio frequency ablation therapy planning system;

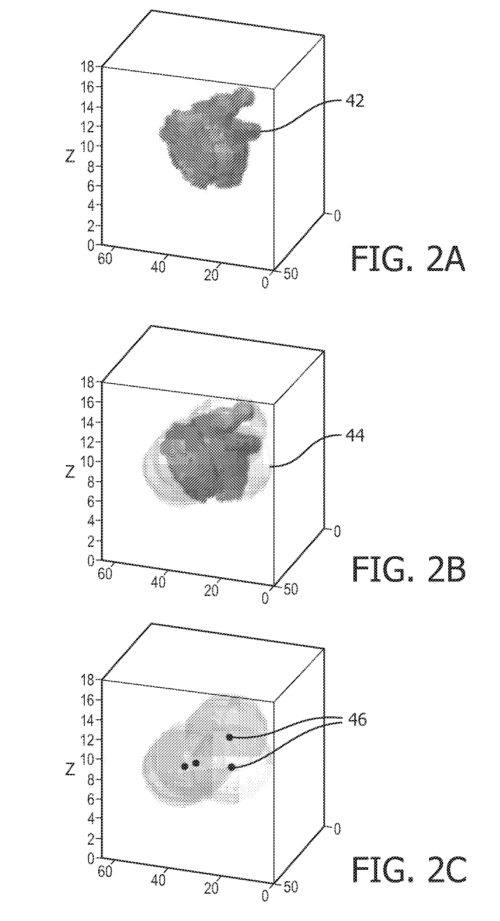

FIGS. 2A-2C illustrate a planned target volume (PTV) and planned spherical ablation zones which cover the PTV and centroids of the corresponding ablation zones, respectively;

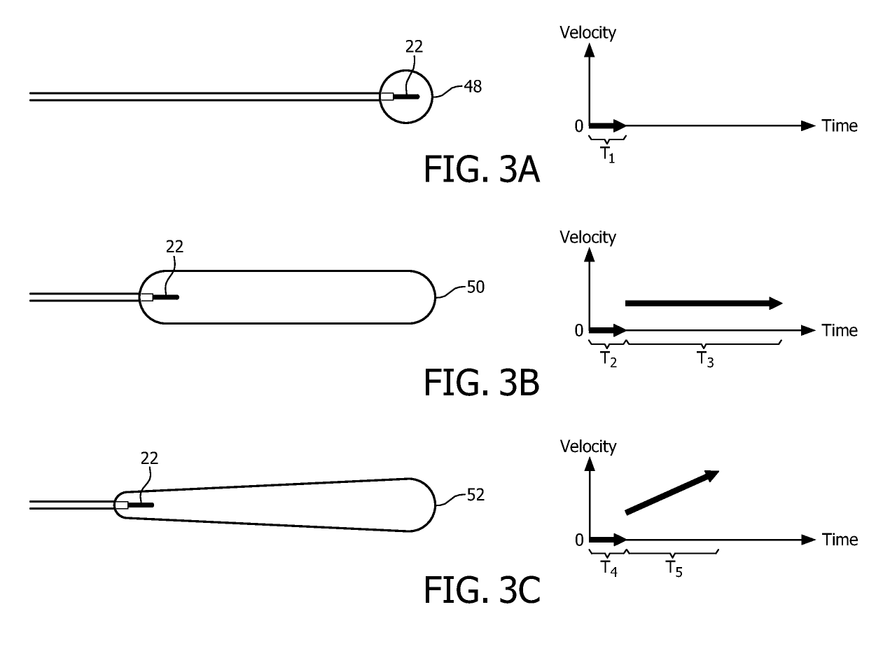

FIG. 3A illustrates a spherical ablation zone, where the velocity is zero over a time period T₁;

FIGS. 3B illustrates a cylindrical, defined by starting with a zero velocity for a time period T₂, then moving the probe at a fixed velocity for a time period T₃;

FIG. 3C illustrates a conical ablation zones defined by starting the ablation with a zero velocity for a time period T₄ (for the same diameter), then moving the probe at an increasing velocity (i.e. positive acceleration) for a period of time T₅;

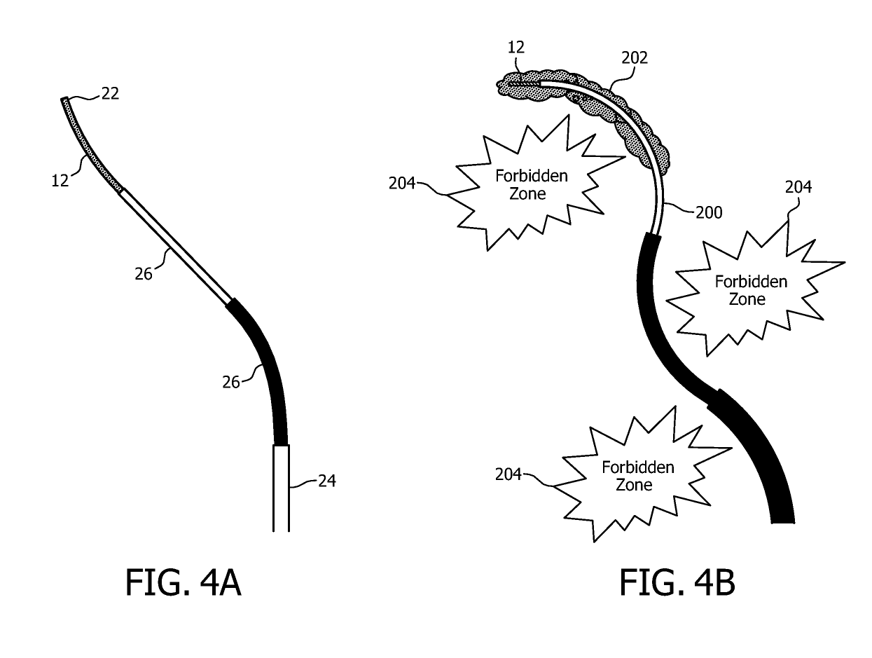

FIG. 4A illustrates an interventional device, a catheter, with multiple nested cannulas and a non-linear ablation probe;

FIG. 4B illustrates a nested cannula retracting as it contains an ablation probe, along the shape of a tumor, around several forbidden zones;

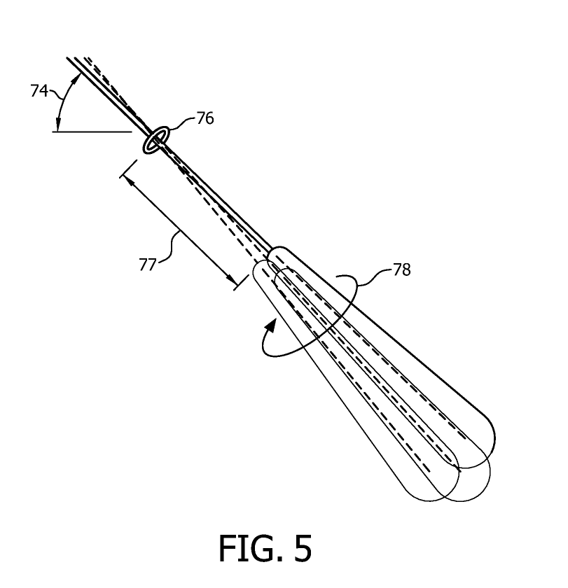

FIG. 5 illustrates an ablation probe demonstrating multiple conical ablation zones that can be achieved by considering various rotations and orientations; and

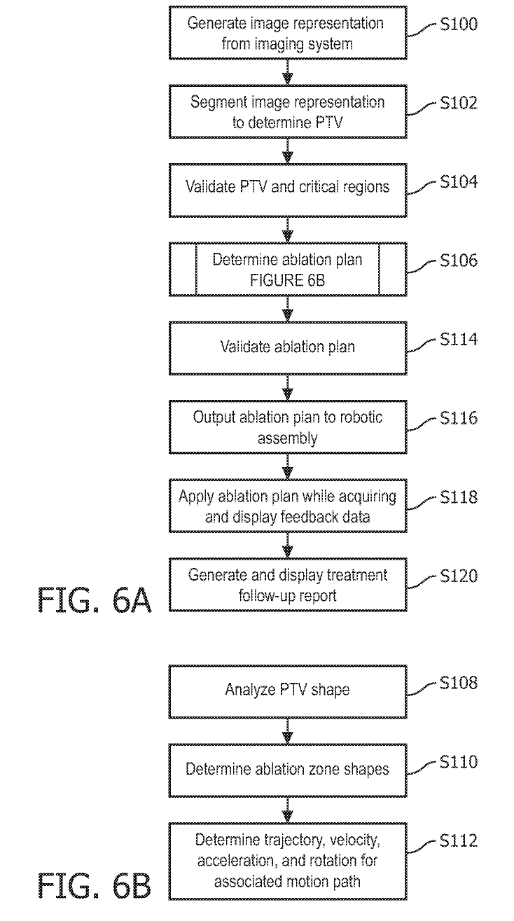

FIGS. 6A and 6B illustrate a methodology for generating an ablation therapy plan.

With reference to FIG. 1, an interventional ablation therapy planning system 10 is illustrated. The ablation planning system 10 facilitates generating a quantitative plan for performing or more ablation protocols to treat a tumor mass or lesion in a patient. Planning includes precisely determining position(s) of an ablation probe and generating ablation zones, or shapes, such that no portion of the tumor mass is left untreated and in order to maximize the amount of the tumor ablated within a fixed procedure time. The system 10 generates a quantitative ablation plan, including target positions, orientations, and motion paths for each ablation zone. The plan is generated to minimize the number of ablations required to treat the entire tumor mass by utilizing ablation shapes that are generated while the probe is under motion to maximize coverage. The generated ablation plan also identifies the entry point or points outside the body that leads to the target volume(s). The ablation can be carried out using a robotic assembly and/or by using image guidance, such as by tracking the position of the ablation probe.

The system 10 includes an ablation probe 12 that is operatively connected to an ablation planning system 14. In the illustrated embodiment, the ablation probe 12 is operatively connected to a power source 16 and an RF generator 18 as well as any suitable component to facilitate the delivery of RF ablation therapy sufficient to kill tumor cells. The RF ablation energy acts to heat the adjacent tissue to approximately 50 degrees causing the cells to break up and thus killing the cells. Under these conditions, there is almost instantaneous cellular protein denaturation, melting of lipid bi-layers and destruction of DNA, RNA, and key cellular enzymes. Alternatively, other therapeutic techniques such as cryo-therapy, electrocautery, high intensity focused ultrasound, radiation, high dose radiation, or the like are also contemplated. The RF ablation probe 12 includes at least one electrode 20 which transmits energy to adjacent tissue to induce hyperthermia. The probe may also include a temperature sensor 22, such as a thermistor, infrared thermometer, thermocouple, or the like, which monitors the target volume temperature during therapy. In another embodiment, the imaging system provides thermographic data, e.g. MRI-based thermometry, infrared thermometry, or the like.

The ablation probe 12 is delivered to the target via an interventional instrument 24 such as a catheter or a scope (e.g. bronchoscope, laparoscope, sigmoidoscope, colonoscope, or the like). At least one nested cannula 26 may be used to navigate complex anatomy to deliver the ablation probe 12 proximate to the target volume. The nested cannula 26 may be constructed from a flexible material such as a polycarbonate plastic, Nitinol, or the like and can be deployed or retracted from a stiffer out sheath. The cannula(s) can be designed prior to therapy according to planning images. The system 10 includes an imaging system 30 such as a computed tomography (CT) scanner. Alternatively, the system 10 may include other imaging modalities such as ultrasound, x-ray fluoroscopy, magnetic resonance imaging (MRI), positron emission tomography (PET), single proton emission tomography (SPECT), or the like. In another embodiment, the system 10 includes multiple imaging modalities to further refine the ablation plan or to provide intra-operative feedback. Combinations of imaging modalities may include any one of the aforementioned imaging modalities. The imaging system 30 generates data which is reconstructed by an imaging processor 32 into a three dimensional (3D) image representation and then stored in a memory unit 34. Objects such as lesions, organs, critical regions can be automatically or semi-automatically segmented by a segmentation unit 36. Segmentation algorithms including object detection, edge detection, or the like are stored in the memory unit 34 and carried out by the imaging processor 36. In another embodiment, a clinician may segment or supplement the machine segmentation object by hand using drawing tools on a graphical user interface (GUI) 38. The segmentation of various regions is used to generate a planned target volume (PTV) which describes a volumetric region intended for full coverage. The PTV is generally a tumor volume plus a margin, typically 1 cm. The PTV is presented to a clinician via the GUI 38 for verification and validation where they may adjust boundaries of objects, classify critical regions, or set the margin to define a larger/smaller PTV. The margin acts to compensate for possible variations and/or errors during therapy. Sources of variation and/or error include unresolved microscopic tumor cells often found surrounding the tumor mass, patient motion, imaging resolution, imaging artifacts, discretization error that affects quantitative planning, tumor boundary uncertainty, non-uniform thermal delivery (e.g. due to non-uniform blood flow dynamic), and the like.

A planning processor 40 of the planning unit 14 analyzes the data associated with the PTV, particularly the dimensions, location, and proximate organs or critical regions, and determines a set of ablation zones for a given ablation probe. Each ablation zone has a predefined ablation volume as shown in FIGS. 2A-2C which illustrate a

PTV 42, planned spherical ablation zones 44 that cover the PTV, and centroids 46 of the corresponding spherical ablation zones 44, respectively. As illustrated in FIG. 2C, the minimum number of ablations necessary to cover the entire target volume issue with congruent ablation zones can be large. To reduce the number of ablation zones, the planning processor algorithmically determines non-congruent, asymmetrical, and/or compound ablation zones which are generated by continuously or intermittently moving the ablation probe along points of a predetermined motion path to entirely cover the PTV. For example, with reference to FIG. 3A, a typical spherical or ellipsoidal ablation zone 48 is generated with a stationary ablation probe. Alternatively, with reference to FIG. 3B, by translating the probe, either distally or proximally, following a velocity profile with a substantially constant velocity, an extruded sphere ablation zone 50 is generated. FIG. 3C illustrates the translation of the probe with a positive velocity profile to generate a conical ablation zone 52. Other shapes of ablation zones are also contemplated such as prolate/oblate spheroids, paraboloids, hyperboloids, or other shapes with varying diameter can be realized by a non-constant velocity.

In one embodiment, the ablation probe delivers ablation therapy unidirectionally such as in the case of focused RF or focused ultrasound energy, instead of omni-directionally as illustrated in FIGS. 3A-3C. Complex ablation zones can be modulated by rotating the probe, in addition to translating the probe forwards or backwards, to generate shapes such as pies, hemispheres, helixes, or the like.

In another embodiment, with reference to FIG. 4A, the ablation probe 12 is not straight. The probe 12 may have a fixed, changeable, or deformable curvature and/or torsion to modulate various ablation zones. The ablation probe can be constructed with an elastic sheath, such as Nitinol or the like, which acts on the probe to create the curvature or torsion. The probe can also be retracted or deployed from a stiffer outer sheath in a controlled manner to generate the contemplated ablation shapes. In another embodiment, the ablation probe 12 is a steerable needle with a rotating, beveled tip which can be externally controlled by a servomechanism or servo.

In another embodiment, with reference to FIG. 4B, a nested cannula 200 delivers a straight RF ablation probe 12 along a desired path such that it covers the full PTV 202, around several forbidden zones 204, e.g. the predetermined critical regions and/or organs. The active portion of the ablation probe 12, remains a fixed length as the enclosing tubes are retracted from smallest to largest, essentially dragging the distal ablation probe 12 along the desired path. Once the PTV 202 is covered, the ablation device can be turned off as the device is retracted from the body. Typically, tumors and PTVs are ablated from the farthest location toward the exit, so that once tissue is ablated, the probe does not retrace through the tissue, risking accidental contamination of tumor cells.

Returning to FIG. 1, in one embodiment, the planning processor 40 algorithmically determines the shape of the ablation zones using shape analysis to determine which geometric shapes fit together to cover the PTV and scales the size of the shapes if necessary. The geometric shapes available for the associated ablation probe are stored in a shape descriptor database stored on a memory unit 60 along with the shape analysis algorithm. Each ablation zone has an associated motion path, which is defined by a trajectory made up of a plurality of points which the ablation probe 12 follows, an acceleration of the probe along the trajectory, and a rotation of the probe during the path. Each motion path is stored in the memory unit 60 as a look-up table with each entry linked to a corresponding ablation zone. The planning processor 40 generates a list of point coordinates for each motion path the probe 12 is to follow according to the shape, orientation, and size of the determined ablation zones.

Alternatively, the planning processor 40 can algorithmically determine the motion path for the determined ablation zone according to the geometric characteristics of the ablation zone shape, e.g. volume, axes, centroid, curvature, angle, etc. There is an entire class of ‘coverage algorithms’ or may use different shapes rather than an ellipsoid as described in prior application [(WO/2008/090484) RF ABLATION PLANNER and publication: “Automated RFA planning for complete coverage of large tumors”, Proc. SPIE, Vol. 7261, 72610D (2009); doi:10.1117/12.811593. The optimal motion paths are iteratively determined by exploiting all available a priori knowledge about the physiology and morphology of the target volume and surrounding tissue to provide the optimal motion paths for each individual patient and probe 12. Once the point coordinates for the ablation zone(s) have been determined, the ablation plan is output to the GUI 38 for approval by the clinician.

In another embodiment, the clinician assembles the various shapes and sizes of available ablation zones to achieve the desired coverage of the PTV. The clinician receives feedback via the GUI 38 regarding the percent coverage achieved and the locations and percent of uncovered regions.

The ablation plan can be carried out manually by a clinician aided by the GUI 38 and a tracking system, mechatronically by a robotic assembly 70, or a combination of robotic guidance and manual control. With reference to FIG. 5, the angle of entry and point of entry of the interventional device 24 are determined by the planning unit or inputted by the clinician then communicated to a robotic controller 72. The robotic controller 72 controls sub-components of the robotic assembly 70 which also provide feedback to the controller 72 regarding the positions of the sub-components. The sub-components servo or guide the angle of entry 74, the point of entry 76, insertion/retraction depth 77, a rotation 78, and rate thereof of the interventional device 24. The robotic assembly also servos or guides the insertion/retraction depth, rotation, and rate thereof of any nested cannula along with that of the ablation probe. The patient anatomy, of course, is registered to the interventional device 24 and ablation probe 12 using known registration methods.

Aspects of the ablation plan, such as the shape of an ablation zone, the motion path, PTV, or the like, can be adjusted in real-time during the procedure based on feedback data using a control loop carried out by the robotic controller 72. The feedback data is a composite of functional, positional, and/or performance data. The functional data is based on the individual patient physiology and acts to update the ablation plan based on changes in the operating environment. The functional data can be based on blood perfusion, blood pressure, cardiac rate, respiratory rate, temperature, tissue impedance, or other physiological parameters which may affect the interventional procedure. For example, the patient's blood flow acts as a heat sink drawing heat away from the target volume which can leave portions of the PTV untreated because the target temperature of approximately 50 degrees centigrade is not maintained during application of each ablation zone. Monitoring changes in local perfusion using known methods, such as MRI, Doppler laser or ultrasound, or the like, allows the planning system to react to changes in the blood flow which may result in a rise or drop in temperature. To account for temperature changes during the intervention, the planning processor 40 may increase/decrease the power output of the power source 16, the frequency of the RF generator 18, and/or the velocity of the RF probe 12 along the prescribed motion path. Additionally, thermodynamic simulations using Finite Element Modeling (FEM), e.g., may be used by the planning processor 40 prior to treatment to describe liquid or gas flow to estimate cooling effects of a nearby heat sink, such as an artery, vein, lung, or the like.

The positional data is based on the position of the interventional instrument 24 including any nested cannulas 26 and the probe 12 relative to the PTV and patient anatomy. Accordingly, a tracking unit 62 compares the current position of the probe 12 to the expected position and, if not, the planning processor 40 adjusts the ablation plan, namely the current motion path, to maneuver the probe to the expected position. If any position along the motion path is omitted, interrupted, or ablation has failed, the position is recorded and revisited as the next position on the motion path or after the remaining points have been ablated.

The positional data can be generated in real-time using imaging techniques such as those described for the planning stage, or a separate imaging modality can be used. For example, MRI or CT can be used to for planning the ablation therapy, while PET, ultrasound, fluoroscopy, or the like can be used to generate the real-time positional data as well as intra-procedural ablation progress. It should be appreciated that other imaging modalities and combination thereof are also contemplated and can be chosen based on the target volume, e.g. severity or extent of malignancy for a tumor. By monitoring the position of the PTV and the interventional instrument 24, nested cannulas 26, and probe 12, the planning processor 40 can detect if the probe has arrived at a first point of a motion path and initiate the ablation plan accordingly. Additionally, the processor 40 can adjust a motion path along which the probe is currently travelling to account for any changes in position resulting from patient movement, clinician error, planning error, or the like. If the position changes beyond a pre-determined limit, the planning processor can terminate the ablation plan.

In another embodiment, the robotic assembly 70 can be used to determine the end point of a motion path by reporting the translation and rotation of each controllable sub-component, which can be combined mathematically by kinematics to determine the location of the tip of the device.

In another embodiment, an electromagnetic system is used to track the probe 12 by providing absolution location relative to a ‘field generator’ which can then be registered to the patient's anatomy, the robotic assembly 70, and/or imaging system 30. Electromagnetic, or active markers, are fitted to the ablation probe 12, nested cannula(s) 26, and/or interventional device 24.

The performance data is based on the performance of the ablation delivery system. The performance data is generated in real-time by monitoring the power output of the power source 16, the output frequency of the RF generator 18, the measured temperature of the temperature sensor 20, changes in impedance of the probe 12, or the like. For example, a sudden drop in local temperature proximate the ablation probe 12 could result from a nearby heat sink. Accordingly, the rates of probe movement or dwell times are adjusted to ensure that every point along the motion path is brought to the target temperature, i.e. the entire ablation zone(s) and PTV is treated.

The planning system 10 offers the advantage of reducing the number of ablations and, more importantly, improving ablation coverage of PTV by planning ablation therapy using asymmetric and/or non-congruent ablation zones and using feedback data to dynamically control ablation. With reference to FIG. 6A, first an image representation of the target volume, e.g. a cancerous lesion, is acquired (S100) using the imaging system 30. The segmentation unit 36 automatically or semi-automatically segments and delineates the target volume and critical structures (S102). The planned target volume (PTV), which includes the segmented target volume plus the margin, and critical structures may be presented to the clinician via the GUI 38 for validation and adjustments if necessary (S104).

Once the PTV and critical structures have been identified and validated the planning processor 40 determines the ablation plan (S106). With reference to FIG. 6B, the planning processor analyzes the shape of the PTV and surrounding anatomy (S108) and determines the set of ablation zones (S110) for the given ablation probe. The points which define the corresponding motion paths and which govern the velocity, acceleration, and/or rotation of the probe 12 along the motion path are determined according to the ablation zone shape, size, orientation, and/or location (S112).

After the ablation plan is generated, it is output to the GUI 38 and visualized on a display unit 90 prior to treatment for validation by the clinician S114. Aspects of the ablation plan are available to the clinician for adjustment and/or validation using an input device 92, such as a keyboard and mouse or the like, via the GUI 38. These aspects may include the determined PTV, ablation zone shapes, motion paths, critical structures, heat sinks, entry point, entry path, initial positions, or the like. Planning processor 40 can also generate multiple ablation plans from which clinician can choose the best plan. Optionally, the planning processor 40 can provide warnings based on information related to proximity to critical, at-risk structures or possible heat sinks. Alternatively, the planning processor can algorithmically chose the optimal ablation plan given a set of boundary conditions determined by the clinician and the patient physiology and/or morphology.

In another embodiment, the ablation plan is determined with little or no user intervention. Non-specific models of the target volume which incorporate a priori knowledge regarding the patient are adapted based on the planning image representations. The processor 40 then generates the optimal ablation plan according to the model of the planned target volume.

The determined ablation zones and corresponding motion paths are outputted to a tracking unit 62 for real-time feedback control of the robotic assembly 70 (S116). The tracking is based the feedback data to create a control loop that governs the velocity, position, and/or rotation of the ablation probe 12. The planning processor uses the feedback data to control the power source 16 and RF generator 18. The ablation plan is then initiated and feedback data is acquired during the procedure (S118). The feedback data can be visualized in real-time on the display 90 of the GUI 38 for the clinician to monitor the progress of the procedure. In this manner, the clinician is able to pause and alter the ablation plan or terminate the plan altogether. Examples of visualized feedback data may include an overlay of the current probe position versus the expected position, local temperature, percent completion, displaying a virtual ablation zone versus the actual ablated region, or the like.

After the ablation plan has completed, a follow-up report based on the ablation plan is generated (S120). A follow-up imaging scan of the treated region is performed. The follow-up report may incorporate an image representation of the actual treatment results fused with an image representation of the ablation plan and/or the acquired feedback data to give the clinician qualitative and quantitative data which can be useful for modifying future ablation plans. Namely, the follow-up report displays a virtual representation of PTV actually treated overlaid with a virtual representation of the PTV expected to be treated. Other feedback data displayed on the follow-up report may include a temperature map, probe 12 positions, thermodynamic simulations, ablated critical, at-risk structures, motion paths, actual/expected ablation zones, or the like.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A method for interventional ablation therapy planning, comprising:
generating an image representation of a target volume in a subject;
determining a planned target volume to receive ablation therapy from an ablation probe, the planned target volume defines a region which includes the target volume;
generating an ablation plan with one or more ablation zones which covers the entire planned target volume with ablation therapy, each ablation zone having a predefined ablation volume, the predefined ablation zone being defined by moving an ablation probe during therapy.

2. The method according to claim 1, further including:
positioning the ablation probe proximate to the target volume at an initial position defined by the generated ablation plan;
applying ablation therapy to the target volume according to the generated ablation plan including moving the ablation probe along a non-stationary motion path.

3. The method according to claim 1, wherein each non-stationary motion path includes:
a trajectory along which the ablation probe travels during application of ablation therapy; and
a velocity and/or acceleration at which the ablation probe travels along the associated trajectory.

4. The method according to claim 2, further including:
at least one of robotically guiding or controlling at least one of the trajectory, acceleration, and rotation of the ablation probe along the non-stationary motion path.

5. The method according to claim 2, further including:
receiving feedback data during application of ablation therapy, the feedback data including at least one of functional data of the subject, positional data of the probe, and performance data of the ablation plan; and
adjusting the at least one of position, velocity, or acceleration of the ablation probe for the corresponding non-stationary motion path in accordance with on the acquired feedback data.

6. The method according to claim 5, wherein
the functional data is based on at least one of blood perfusion, blood pressure, cardiac rate, respiratory rate, temperature, and tissue impedance;
the positional data is based on at least one of position of the ablation probe relative to the PTV, the positional data is acquired via the imaging system; and
the performance data is based on the power output, frequency, temperature, and impedance of the probe.

7. The method according to claim 2, further including:
prior to the application of ablation therapy, validating the generated ablation plan;
during application of ablation therapy in accordance to the validated ablation plan, displaying the feedback data in real-time; and
after application of ablation therapy, displaying a follow-up report according to an actual ablated volume and the PTV.

8. The method according to claim 1, wherein the predefined ablation volume includes at least one of:
an elongated tubular volume created by moving the ablation probe at a substantially constant velocity during ablation;
a generally conical volume created by accelerating and/or decelerating the ablation probe during ablation;
a helical volume created by moving and rotating a curved ablation probe during ablation;
a prolate/oblate spheroid volume created by decelerating then accelerating the ablation probe during ablation;
a hyperboloid volume created by accelerating then decelerating ablation probe during ablation; and
a hemispherical volume created by rotating a focused ablation probe during ablation.

9. An interventional ablation therapy planning system, comprising:
an imaging system which generates an image representation of a target volume in a subject;
a segmentation unit which determines a planned target volume to receive ablation therapy, the planned target volume defines a region which includes the target volume;
a planning processor which generates an ablation plan with one or more ablation zones which cover the entire planned target volume with ablation therapy, each ablation zone has a predetermined ablation volume, the predetermined ablation zone being defined by moving an ablation probe during ablation.

10. The interventional ablation therapy planning system according to claim 9, further including:
an interventional device which positions the ablation probe proximate to the target volume at an initial position defined by the generated ablation plan; and
an ablation source which applies ablation therapy to the target volume according to the generated ablation plan as the ablation probe moves along a non-stationary motion path.

11. The interventional ablation therapy planning system according to claim 10, wherein each non-stationary motion path includes:
a trajectory along which the ablation probe travels during application of ablation therapy; and
a velocity and/or acceleration at which the ablation probe travels along the associated trajectory.

12. The interventional ablation therapy planning system according to claim 10, further including:
a robotic assembly which guides and/or controls at least one of the position, velocity, acceleration, and rotation of the ablation probe along the non-stationary motion path.

13. The interventional ablation therapy planning system according to claim 10, further including:
a tracking unit which receives feedback data during application of the ablation therapy, the feedback data including at least one of functional data of the subject, positional data of the probe, and performance data of the ablation plan; and
a robotic controller which adjusts the at least one of position, velocity, or acceleration of the ablation probe in accordance with the acquired feedback data.

14. The interventional ablation therapy planning system according to claim 13, wherein
the functional data is based on at least one of blood perfusion, blood pressure, cardiac rate, respiratory rate, temperature, and tissue impedance;
the positional data is based on at least one of position of the ablation probe relative to the PTV, the positional data is acquired via the imaging system; and
the performance data is based on the power output, frequency, temperature, and impedance of the probe.

15. The interventional ablation therapy planning system according to claim 9, further including:
a graphical user interface for validating the generated ablation plan prior to the application of ablation therapy, displaying feedback data in real-time during the application of ablation therapy in accordance to the validated ablation plan, and displaying a follow-up report according to an actual ablated volume and the PTV.

16. The interventional ablation therapy planning system according to claim 9, wherein
the ablation probe is nested within a cannula of the interventional device, at least one of the ablation probe and cannula are steerable.
the robotic assembly controls at least one of an insertion point, position, and orientation of the interventional device.

17. The interventional ablation therapy planning system according to claim 9, wherein the planning processor includes a memory which stores a plurality of the predetermined ablation volumes.

18. The interventional ablation therapy planning system according to claim 9, wherein the predefined ablation volume includes at least one of:
an elongated tubular volume created by moving the ablation probe at a substantially constant velocity during ablation;
a generally conical volume created by accelerating and/or decelerating the ablation probe during ablation;
a helical volume created by moving and rotating a curved ablation probe during ablation;
a prolate/oblate spheroid volume created by decelerating then accelerating the ablation probe during ablation;
a hyperboloid volume created by accelerating then decelerating ablation probe during ablation; and
a hemispherical volume created by rotating a focused ablation probe during ablation.

19. A method for generating an ablation zone using an ablation probe, comprising:
determining a trajectory of the ablation probe;
determining a non-constant velocity profile of the ablation probe along the determined trajectory; and
applying ablation therapy while the ablation probe travels along the determined trajectory at the determined non- constant velocity profile.

20. The method according to claim 19, wherein the profile of the ablation probe is at least one of linear or non-linear.
